(19) United States
(12) Patent Application Publication
(10) Pub. No.: US 2022/0333108 A1
(43) Pub. Date: Oct. 20, 2022

(54) COMBINATIONS OF SIRNAS WITH SIRNAS AGAINST SULF2 OR GPC3 FOR USE IN TREATING CANCER

(71) Applicant: Sirnaomics, Inc., Gaithersburg, MD (US)

(72) Inventors: David EVANS, Gaithersburg, MD (US); John XU, Gaithersburg, MD (US); Patrick Y. LU, Gaithersburg, MD (US); Vera SIMONENKO, Gaithersburg, MD (US)

(21) Appl. No.: 17/711,975

(22) Filed: Apr. 1, 2022

Related U.S. Application Data
(60) Provisional application No. 63/169,564, filed on Apr. 1, 2021.

(51) Int. Cl. C12N 15/113; A61P 35/00

(52) U.S. Cl. CPC C12N 15/113; A61P 35/00

(57) ABSTRACT

Compositions and methods are provided for silencing the Sulf2 and/or GPC3 genes in vivo. Potent siRNA sequences are provided that silence regions of the Sulf2 and GPC3 genes that are identical in human, mice and non-human primates. Combinations of siRNAs also are provided that result in additivity or synergy with silencing of Sulf2 and/or GPC3. Silencing SULF2+TGFβ1 showed a dramatic effect against cancer growth in vitro and in vivo.

### RELATED APPLICATION

This application claims priority to provisional application No. 63/169,564 filed Apr. 1, 2021, which is incorporated herein by reference in its entirety.

### BACKGROUND

Human sulfatase 2 (Sulf2) functions as an oncoprotein in hepatocellular carcinoma (HCC) development by promoting tumor growth and metastasis via enhancement of fibroblast growth factor-2/extracellular signal-regulated kinase and WNT/β-catenin signaling (Zheng et al. Genes Chromosomes Cancer. 52(3): 225-236 (2013)). Furthermore, Sulf2 activates the transforming growth factor beta (TGFB) and Hedgehog/GLI1 pathways in HCC (Id.).

Sulf2 is an extracellular heparan sulphate 6-O-endosulphatase. It has an oncogenic effect in hepatocellular carcinoma (HCC) that is partially mediated through glypican 3 (GPC3) through increased heparin-binding growth factor signaling and HCC cell growth (La et al., Liver Int. 30(10): 1522-1528 (2010)). Sulf2 increases phosphorylation of the anti-apoptotic Akt kinase substrate GSK3β and Sulf2 expression is associated with a decreased apoptotic index in human HCCs (Id.). Furthermore Glypican 3 has been proposed to be a valid biomarker and a therapeutic target for treatment of HCC (Wang et al., Hepatobiliary Pancreat Dis Int;14:361-366 (2015)). Furthermore, SULF enzymes promote key signaling pathways by mobilizing protein ligands (e.g., Wnt, GDNF, PDGF-B, BMP-4) from HSPG sequestration, thus liberating the ligands for binding to signal transduction receptors (Morimoto-Tomita et al., J Biol Chem 277: 49175-49185 (2002)). Sulf2 has been directly implicated as a driver of carcinogenesis in NSCLC (Lemjabbar-Alaoui et al., supra), murine and human malignant glioma including glioblastoma and oligodendroglioma (Johansson et al., Oncogene 24: 3896-3905 (2005); Phillips et al., J. Clin Invest 122: 911-922 (2012)), pancreatic cancer (Nawroth et al., PLoS ONE 2: e392), and hepatocellular carcinoma (Lai et al., Hepatology 52: 1680-1689 (2010)) as well as Head and Neck Cancer (et al., Oncotarget 7 (28): 43177-43187 (2016)).

Quantitative PCR or gene microarray analyses have reported the overexpression of SULFs in a wide range of human tumors: hepatocellular carcinoma (HCC) (Lai et al., Gastroenterology; 126:231— 248 (2004)), pancreatic (Li et al., Mol Cancer. 4:14 (2005)), head and neck squamous cell carcinoma Kudo et al., Cancer Res. 66:6928-6935 (2006)., gastric cancer (Junnila et al., Genes Chromosomes Cancer. 49:28-39 (2010)), lung adenocarcinoma (Lemjabbar-Alaoui et al., Oncogene; 29:635-646 (2010)), and lung squamous cell carcinoma (Id.) for SULF1; and hepatocellular carcinomas (Lai et al., Hepatology, 47:1211— 1222 (2008)), lung adenocarcinoma, and lung squamous cell carcinoma (Lemjabbar-Alaoui et al., supra) and pancreatic cancer (Atha san et al., British Journal of Cancer 115: 797-804 (2016)) for Sulf2

In NSCLC it was found that: 1) upregulation of both SULFs occurred at the transcript level; 2) Sulf2 protein expression occurred in 20 of 20 human NSCLC tumors compared to minimal levels in normal lung; 3) Sulf2 protein promotes the in vitro malignant phenotype, and the tumorigenicity in mice of SULF-2 positive human NSCLC cell lines; and 4) Sulf2 promotes human lung carcinogenesis by regulation of Wnt signaling and the kinase activity of three critical receptors (i.e., EGFR, IGF-1R and cMet) (Lemjabbar-Alaoui et al., supra). Dysregulation of each of these three receptors has been causally linked to lung cancer development, progression, and increased resistance to chemotherapy (Engelman et al., Clin. Cancer Res. 14: 2895-2899 (2008); Engelman et al., Science 316: 1039-1043 (2007); Lei et al., Anticancer Res. 19: 221-228 (1999)).

Glypican-3 (GPC3) is a member of the heparan sulfate proteoglycan family and is widely expressed on various cell surfaces of embryos. In adults, GPC3 is normally expressed only in the ovary, but is specifically expressed in liver cancer tissues, and presents as soluble GPC3 (sGPC3) in peripheral blood of HCC patients. GPC3 expression does not occur in liver tissues of healthy adults. Prior studies have indicated that silencing GPC3 using siRNA or shRNA can produce a therapeutic effect against HCC (Ruan et al., Int. J. Mol. Med. 28:497-503 (2011); Miao et al., J. Cell Biochem.;114:625-631 (2013)), However, each of the studies only used a single siRNA or shRNA to demonstrate efficacy.

### SUMMARY OF THE INVENTION

siRNA molecules are provided that target Sulf2 expression, where the sequence targets a Sulf2 sequence common to humans and mice. The molecules may have, for example, the sense strand of SEQ ID NO:1-9.

Also provided are siRNA molecules that target GPC3 expression, where the sequence targets a GPC3 sequence common to humans and mice. The molecules may have, for example, the sense strand of SEQ ID NO:10-31.

Pharmaceutical compositions containing an siRNA molecule, or combinations of siRNA molecules, as described above also are provided. The pharmaceutical compositions may contain one or more molecules as described above, together with at least one siRNA that targets TGFβ1, FGFR, β-Catenin, GPC3, Yap1, MET or TERT. The siRNA that targets TGFβ1, FGFR, β-Catenin, Yap1, MET, or TERT may be selected from the molecules having the sense strand of SEQ ID NOs:32-37.

The pharmaceutical composition as described above may be a nanoparticle composition, such as a composition containing HKP. In particular embodiments the HKP may be HKP(+H).

Further provided are methods of treating cancer in a subject, such as a human subject, that include administering to the subject an effective amount of a molecule or pharmaceutical composition as described above. In specific embodiments the method may include administering to the subject an effective amount of a small molecule chemotherapeutic drug. The small molecule therapeutic drug may be a protein kinase inhibitor, such as sorafenib. The cancer may be, for example, hepatocellular carcinoma, cholangiocarcinoma, pancreatic cancer, lung cancer, colon cancer, head and neck cancer or esophageal cancer. The molecules or compositions as described above may be is administered systemically or intratumorally.

### BRIEF DESCRIPTION OF THE DRAWINGS

FIG. 1 shows silencing of Sulf2 in HepG2 cells by the Sulf 2_1 sequence.

FIG. 2 shows silencing of Sulf2 in SKHepl cells by the Sulf 2_1 sequence.

FIG. 3 shows the dose response effect of the siRNA Sulf_1 sequence in silencing Sulf2 in HepG2 cells at 48 h exposure.

FIG. 4 shows the potential signaling mechanism between Sulf2 and GPC3.

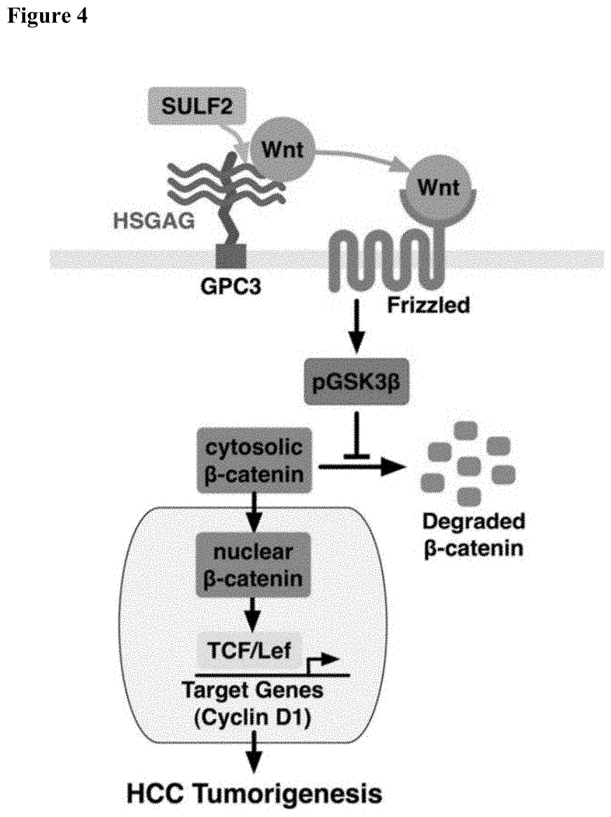

FIG. 5 shows how Sulf2 silencing at 3 nM is as potent as Sorafenib treatment at 4 μM in HepG2 HCC cells incubated with the siRNAs for 96 h.

FIG. 6 shows the effects of combinations of siRNAs on efficacy against Hep3B cells in vitro.

FIG. 7 shows the effects of combinations of siRNAs on efficacy against HepG2 cells in vitro.

FIG. 8 shows inhibition of TERT by TERT siRNA in HepG2 cells.

FIG. 9 shows the effect of Sulf 2 siRNA together with additional siRNAs on viability of pancreatic cancer cells (Capan2 and BxPC3). FIG. 9A shows the effect of combination of siRNAs on viability of Capan2 cells and FIG. 9B shows that the combination of Sulf2 siRNA together with TGFB1 siRNA provided the greatest response in BxPC3 cells.

FIG. 10 shows the efficacy of Sulf2/TGFb1 siRNA delivered intratumorally into pancreatic cancer xenograft tumor at 1mg/Kg.

FIG. 11 shows the effect of administration of siRNAs on the weight of tumor recovered from treated animals.

FIG. 12 shows the effect of coadministration of TGFβ1/Sulf2 siRNA on TGFβ1 mRNA expression in the tumor samples recovered.

FIG. 13 shows that Sulf2+TGFb1 siRNA treatment resulted in a marked reduction in tumor size.

### DETAILED DESCRIPTION

Compositions and methods are provided for silencing the Sulf2 and/or GPC3 genes in vivo. Specifically, potent siRNA sequences are provided that silence regions of the Sulf2 and GPC3 genes that are identical in human, mice and non-human primates. These siRNAs are 25-mer blunt ended double stranded RNAs.

Also provided are compositions and methods for silencing genes that, when silenced along with Sulf2 or GPC3, exhibit improvement in efficacy compared with either treatment alone. A nanoparticle delivery agent is used to deliver these combinations of two siRNAs to the appropriate tissue in the body (liver for HCC for example) and into the same cell at the same time. This provides an augmented, synergistic effect on inhibition of the growth of the tumor cells compared to either siRNA alone.

Silencing of either Sulf2 or GPC3 genes using the siRNA molecules described herein also potentiates the activity of small molecule kinase inhibitors. It has been shown that sulfatase-2 protects hepatocellular carcinoma cells against apoptosis induced by the PI3K inhibitor LY294002 and ERK and JNK kinase inhibitors (Lai et al., 2010 supra). Also, loss of function of Sulf2—either by mutation (N491K) or inhibition (by siRNAs)—has been shown to enhance sorafenib sensitivity in liver cancer cells and in vivo mouse models. Yoon et al., Oncogene 37:4443-4454 (2018).

Combinations containing two siRNAs, where one siRNA targets SULF2 or GPC3 and a second siRNA targets TGF-β, β-catenin, YAP (yes-associated protein, also known as YAP1 or YAP65), telomerase reverse transcriptase (TERT), MET, or fibroblast growth factor receptor (FGFR). The two siRNAs can be mixed and used to form nanoparticles with an siRNA delivery vehicle, such as HKP or HKP(+H), so that both siRNAs are loaded into the same nanoparticle. When delivered to cells in vitro or in vivo, the nanoparticles release both siRNAs simultaneously —silencing both targets and providing additive or synergistic effects resulting from the silencing of the two targets. Silencing SULF2 and TGFβ1 reduces the growth rate of pancreatic cancer cells, while silencing SULF2 and TERT provides a significant improvement in inhibiting the growth rate of liver cancer cells.

### Selection of siRNA sequences

Multiple siRNAs were screened to identify the most potent sequence. The Sulf2 gene sequences in mice and humans were compared for regions of identity where siRNAs would be predicted to induce optimal silencing. The sense strands of the mouse/human siRNAs are shown below:

S2_1 Rec#, 2, SiRNA SEQ,
(SEQ ID NO: 1)
GCCGCACCTTTGCCGTGTACCTCAA,
S2_2 Rec#, 5, SiRNA SEQ,
(SEQ ID NO: 2)
CCAACATGCTCCAGCGGAAGCGCTT,
S2_3 Rec#, 7, SiRNA SEQ,
(SEQ ID NO: 3)
CCAGAAGAATGTGACTGTCACAAAA,
S2_4 Rec#, 8, SiRNA SEQ,
(SEQ ID NO: 4)
GCTCCAGTCTGCATCCTTTCAGGAA,
S2_5 Rec#, 9, SiRNA SEQ,
(SEQ ID NO: 5)
GGAAGGGCCTGCAAGAGAAGGACAA,
S2_6 Rec#, 10, SiRNA SEQ,
(SEQ ID NO: 6)
GGGCGAAAGTCATTGGAATTTTTAA,
S2_7 Rec#, 11, SiRNA SEQ,
(SEQ ID NO: 7)
CCTCGCAGTTGTGGACATTTCTGTT,
S2_8 Rec#, 12, SiRNA SEQ,
(SEQ ID NO: 8)
GGACATTTCTGTTCCTGTCCAGATA,
S2_9 Rec#, 13, SiRNA SEQ,
(SEQ ID NO: 9)
CCTTTGACATTTTGTAAAAGGCCAT,

siRNA molecules against conserved regions of mouse and human GPC3 also were designed

(SEQ ID NO: 10)
CCCGCCCTCGCCCAGCGCCCAGGTA
(SEQ ID NO: 11)
CCAGCGCCCAGGTAGCTGCGAGGAA
(SEQ ID NO: 12)
CGCCCAGGTAGCTGCGAGGAAACTT
(SEQ ID NO: 13)
GCCCAGGTAGCTGCGAGGAAACTTT
(SEQ ID NO: 14)
CCCAGGTAGCTGCGAGGAAACTTTT
(SEQ ID NO: 15)
CCGGGACCGTGCGCACCGCGTGCTT
(SEQ ID NO: 16)
GCCCAACATGCTGCTCAAGAAAGAT
(SEQ ID NO: 17)
GCTGCTCAAGAAAGATGGAAGAAAA
(SEQ ID NO: 18)
GCTCAAGAAAGATGGAAGAAAAATA
(SEQ ID NO: 19)
GCGGTTTTCCAAGAGGCCTTTGAAA
(SEQ ID NO: 20)
CGGTTTTCCAAGAGGCCTTTGAAAT
(SEQ ID NO: 21)
GGTTTTCCAAGAGGCCTTTGAAATT
(SEQ ID NO: 22)
CCAAGAGGCCTTTGAAATTGTTGTT
(SEQ ID NO: 23)
GGCCTTTGAAATTGTTGTTCGCCAT
(SEQ ID NO: 24)
GCCTGACTCCACAAGCTTTTGAGTT
(SEQ ID NO: 25)
CCTGACTCCACAAGCTTTTGAGTTT
(SEQ ID NO: 26)
CCGAGGAGCAAGACGTGACCTGAAA
(SEQ ID NO: 27)
GGAGCAAGACGTGACCTGAAAGTAT
(SEQ ID NO: 28)
CCCCAAGCTTATTATGACCCAGGTT
(SEQ ID NO: 29)
CCCAAGCTTATTATGACCCAGGTTT
(SEQ ID NO: 30)
GCTCTTACTGCCAGGGACTGATGAT
(SEQ ID NO: 31)
GCAATGTGGTCATGCAAGGCTGTAT

Additional siRNA sequences were selected to target the genes that might synergize with Sulf2/GPC3 silencing to augment activity in inhibiting tumor cell growth. The sense strands of these siRNA molecules are shown below:

(SEQ ID NO: 32)
MET Sequence: GCACUAGCAAAGUCCGAGAdTdT
(SEQ ID NO: 33)
TERT Sequence: CCAUCAGAGCCAGUCUCACCUUCAA
(SEQ ID NO: 34)
YAP sequence: GGUGAUACUAUCAACCAAAdTdT
(SEQ ID NO: 35)
FGFR sequence; CCACCACAUUGACUACUAUdTdT
(SEQ ID NO: 36)
B-Catenin sequence; GGACCUAUACUUACGAAAAdTdT
(SEQ ID NO: 37)
TGFβ sequence: CCCAAGGGCUACCAUGCCAACUUCU

The siRNA molecules may be used as single duplex molecules, or two or more siRNA molecules that target Sulf2 and/or GPC3 may be combined. In addition, an siRNA that binds Sulf2 or GPC3 may be combined with another siRNA that targets a further cancer-associated mRNA. Examples of additional targets include MET, YAP, FGFR4, β-Catenin, TERT and TGFβ1. Exemplary combinations of siRNA molecules include, but are not limited to, siRNAs targeting:
Sulf2 and MET
Sulf2 and YAP
Sulf2 and FGFR4
Sulf2 and β-Catenin
Sulf2 and TERT
Sulf2 and TGFβ1
GPC3 and MET
GPC3 and YAP
GPC3 and FGFR4
GPC3 and β-Catenin
GPC3 and TERT
GPC3 and TGFβ1
SULF2 AND GPC3 and MET
SULF2 AND GPC3 and YAP
SULF2 AND GPC3 and FGFR4
SULF2 AND GPC3 and β-Catenin
SULF2 AND GPC3 and TERT
SULF2 AND GPC3 and TGFβ1

Reference herein to the siRNA molecule of SEQ ID NO:X will be understood to refer to the duplex formed by the sense strand (SEQ ID NO:X) and the corresponding antisense strand.

The siRNA molecules may be formulated in nanoparticles for administration. The nanoparticles may contain one or more lipids, including neutral and cationic lipids. Advantageously, the nanoparticles contain an HKP (histidine-lysine polymer) as described, for example, in U.S. Pat. Nos. 7,163,695, 7,070,807, and 7,772,201, the contents of each of which are hereby incorporated in their entireties. Advantageously, the nanoparticles contain a highly-branched HKP as described in U.S. Pat. No. 7,772,201.

As used herein, silencing a gene means reducing the concentration of the mRNA transcript of that gene such that the concentration of the protein product of that gene is reduced by at least 10%, at least 20%, at least 30%, at least 40%, at least 50%, at least 70%, at least 80% or at least 90% or more.

### Formation of nanoparticles containing siRNAs targeting Sulf2 and/or GPC3.

The siRNA molecules containing the molecules described above advantageously are formulated into nanoparticles for administration to a subject. Various methods of nanoparticle formation are well known in the art. See, for example, Babu et al., IEEE Trans Nanobioscience, 15: 849-863 (2016).

Advantageously, the nanoparticles are formed using one or more histidine/lysine (HKP) copolymers. Suitable HKP copolymers are described in WO/2001/047496, WO/2003/090719, and WO/2006/060182, the contents of each of which are incorporated herein by reference in their entireties. HKP copolymers form a nanoparticle containing an siRNA molecule, typically 100-400 nm in diameter. HKP and HKP(+H) both have a lysine backbone (three lysine residues) where the lysine side chain ϵ-amino groups and the N-terminus are coupled to [KH₃]₄K (for HKP) or KH₃KH₄[KH₃]₂K (for HKP(+H). The branched HKP carriers can be synthesized by methods that are well-known in the art including, for example, solid-phase peptide synthesis.

Methods of forming nanoparticles are well known in the art. Babu et al., supra. Advantageously, nanoparticles may be formed using a microfluidic mixer system, in which an siRNA targeting Sulf2 and/or GPC3 is mixed with one or more siRNAs targeting other proteins and the two siRNAs are mixed together before being formulated with HKP polymers at a fixed flow rate to give nanoparticles of a given size. The flow rate can be varied to vary the size of the nanoparticles produced. A suitable microfluidic mixer is, for example, a NanoAssemblr microfluidic instrument (Precision NanoSystems, Inc.).

### Determination of Efficacy of the siRNA Molecules

Depending on the particular target Sulf2 and/or GPC3 RNA sequences and the dose of the nanoparticle composition delivered, partial or complete loss of function for the Sulf2 and/or GPC3 RNAs may be observed. A reduction or loss of RNA levels or expression (either Sulf2 and/or GPC3 RNA expression or encoded polypeptide expression) in at least 50%, 60%, 70%, 80%, 90%, 95% or 99% or more of targeted cells is exemplary. Inhibition of Sulf2 and/or GPC3 RNA levels or expression refers to the absence (or observable decrease) in the level of Sulf2 and/or GPC3 RNA or Sulf2 and/or GPC3 RNA-encoded protein. Specificity refers to the ability to inhibit the Sulf2 and/or GPC3 RNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target Sulf2 and/or GPC3 RNA sequence(s) by the dsRNA agents of the invention also can be measured based upon the effect of administration of such dsRNA agents upon development/progression of a Sulf2 and/or GPC3 associated disease or disorder, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, or 10⁷-fold reduction in cancer cell levels could be achieved via administration of the nanoparticle composition to cells, a tissue, or a subject. The subject may be a mammal, such as a human.

### Determination of Dosage and Toxicity

Toxicity and therapeutic efficacy of the compositions may be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds advantageously exhibit high therapeutic indices

Data from cell culture assays and animal studies can be used in formulating a range of dosage for use in humans. The dosage of the compositions advantageously is within a range of circulating concentrations that include the ED₅₀ with little or no toxicity. The dosage may vary within this range depending upon the dosage form employed and the route of administration utilized. For the compositions described herein, a therapeutically effective dose can be estimated initially from cell culture assays. A dose may be formulated in animal models to achieve a circulating plasma concentration range that includes the IC₅₀ (i.e., the concentration of the composition which achieves a half-maximal inhibition of symptoms) as determined in cell culture. Such information can be used to more accurately determine useful doses in humans. Levels in plasma may be measured, for example, by high performance liquid chromatography.

A therapeutically effective amount of a composition as described herein can be in the range of approximately 1 pg to 1000 mg. For example, 10, 30, 100, or 1000 pg, or 10, 30, 100, or 1000 ng, or 10, 30, 100, or 1000 μg, or 10, 30, 100, or 1000 mg, or 1-5 g of the compositions can be administered. In general, a suitable dosage unit of the compositions described herein will be in the range of 0.001 to 0.25 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. The pharmaceutical composition can be administered once daily, or may be dosed in dosage units containing two, three, four, five, six or more sub-doses administered at appropriate intervals throughout the day. In that case, the dsRNA contained in each sub-dose must be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

The compositions may be administered once, one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition as described herein may include a single treatment or, advantageously, can include a series of treatments.

As used herein, a pharmacologically or therapeutically effective amount refers to that amount of an siRNA composition effective to produce the intended pharmacological, therapeutic or preventive result. The phrases “pharmacologically effective amount” and “therapeutically effective amount” or “effective amount” refer to that amount of the composition effective to produce the intended pharmacological, therapeutic or preventive result. For example, if a given clinical treatment is considered effective when there is at least a 30% reduction in a measurable parameter associated with a disease or disorder, a therapeutically effective amount of a drug for the treatment of that disease or disorder is the amount necessary to effect at least a 30% reduction in that parameter.

Suitably formulated pharmaceutical compositions as described herein may be administered by means known in the art such as by parenteral routes, including intravenous, intramuscular, intraperitoneal, subcutaneous, transdermal, airway (aerosol), rectal, vaginal and topical (including buccal and sublingual) administration. Advantageously, the pharmaceutical compositions are administered by intravenous or intraparenteral infusion or injection.

### Pharmaceutical Compositions and Methods of Administration

The nanoparticle compositions may be further formulated as a pharmaceutical composition using methods that are well known in the art. The composition may be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, trehalose, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The compositions may also be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Methods of Treatment

The compositions described herein may be used to treat proliferative diseases, such as cancer, characterized by expression, and particularly altered expression, of Sulf2 and/or GPC3 . Exemplary cancers include: hepatocellular carcinoma, esophageal cancer, head and neck cancer, bladder cancer, pancreatic cancer, cholangiocarcinoma, lung cancer (NSCLC, SCLC, LUSC), colon cancer, glioblastoma, breast cancer, gastric adenocarcinomas, prostate cancer, ovarian carcinoma, cervical cancer, AML, ALL, myeloma or non-Hodgkins lymphoma. In these methods the composition may be delivered systemically or intratumorally.

Other cancers include renal cancer (e.g., papillary renal carcinoma), stomach cancer, medulloblastoma, thyroid carcinoma, rhabdomyosarcoma, osteosarcoma, squamous cell carcinoma (e.g., oral squamous cell carcinoma), melanoma, and hematopoietic disorders (e.g., leukemias and lymphomas, and other immune cell-related disorders). Further cancers include bladder, cervical (uterine), endometrial (uterine), head and neck, and oropharyngeal cancers.

The compositions may be administered as described above and, advantageously may be delivered systemically or intratumorally. The compositions may be administered as a monotherapy, i.e. in the absence of another treatment, or may be administered as part of a combination regimen that includes one or more additional medications. Advantageously, the compositions are used as part of a combination regimen that includes an effective amount of at least one additional chemotherapy drug, as described below.

Further provided are methods of treating cancer in a subject, in which the nanoparticle composition as described above is administered together with an effective amount of a chemotherapy drug. Examples of suitable chemotherapy drugs include protein kinase inhibitors, platinum-containing drugs such as cisplatin, oxaloplatin, or carboplatin, docetaxel (Taxotere), gemcitabine (Gemzar), paclitaxel (Taxol), pemetrexed (Alimta),vinorelbine (Navelbine), Abraxane (Paclitaxel Albumin-stabilized Nanoparticle Formulation), Afatinib Dimaleate, Afinitor (Everolimus), Afinitor Disperz (Everolimus), Alecensa (Alectinib), Alectinib, Alimta (Pemetrexed Disodium), Alunbrig (Brigatinib), Atezolizumab, Avastin (Bevacizumab), Bevacizumab, Brigatinib, Capmatinib Hydrochloride, Carboplatin, Cemiplimab-rwlc, Ceritinib, Crizotinib, Cyramza (Ramucirumab), Dabrafenib Mesylate, Dacomitinib, Docetaxel, Doxorubicin Hydrochloride, Durvalumab, Entrectinib, Erlotinib Hydrochloride, Everolimus, Gavreto (Pralsetinib), Gefitinib, Gilotrif (Afatinib Dimaleate), Gemcitabine Hydrochloride, Gemzar (Gemcitabine Hydrochloride), Imfinzi (Durvalumab), Infugem (Gemcitabine Hydrochloride), Ipilimumab, Iressa (Gefitinib), Keytruda (Pembrolizumab), Libtayo (Cemiplimab-rwlc), Lorbrena (Lorlatinib), Lorlatinib, Mekinist (Trametinib Dimethyl Sulfoxide), Methotrexate Sodium, Mvasi (Bevacizumab), Necitumumab, Nivolumab, Opdivo (Nivolumab), Osimertinib Mesylate, Paclitaxel, Paclitaxel Albumin-stabilized Nanoparticle Formulation, Paraplat (Carboplatin), Paraplatin (Carboplatin), Pembrolizumab, Pemetrexed Disodium, Portrazza (Necitumumab), Pralsetinib, Ramucirumab, Retevmo (Selpercatinib), Rozlytrek (Entrectinib), Selpercatinib, Nexavar (sorafenib) Tabrecta (Capmatinib Hydrochloride), Tafinlar (Dabrafenib Mesylate), Tagrisso (Osimertinib Mesylate), Tarceva (Erlotinib Hydrochloride), Taxotere (Docetaxel), Tecentriq (Atezolizumab), Tepmetko (Tepotinib Hydrochloride), Tepotinib Hydrochloride, Trametinib Dimethyl Sulfoxide, Trexall (Methotrexate Sodium), Vizimpro (Dacomitinib), Vinorelbine Tartrate, Xalkori (Crizotinib), Yervoy (Ipilimumab), Zirabev (Bevacizumab), Zykadia (Ceritinib), carboplatin-taxol, gemcitabine-cisplatin, Afinitor (Everolimus), Atezolizumab, Doxorubicin Hydrochloride, Durvalumab, Etopophos (Etoposide Phosphate), Etoposide, Etoposide Phosphate, Everolimus, Hycamtin (Topotecan Hydrochloride), Imfinzi (Durvalumab), Lurbinectedin, Methotrexate Sodium, Nivolumab, Opdivo (Nivolumab), Tecentriq (Atezolizumab), Topotecan Hydrochloride, and Trexall (Methotrexate Sodium).

### EXAMPLES

### Example 1

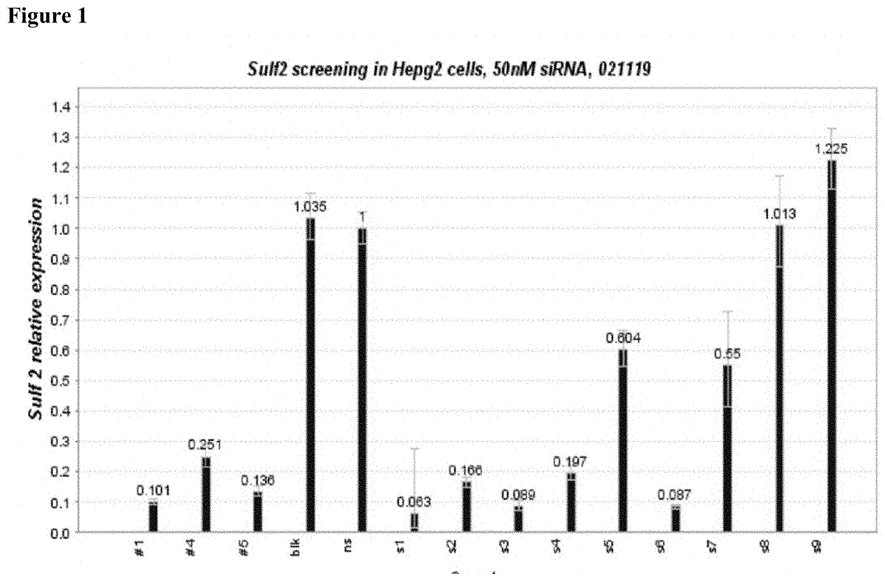

siRNA sequences against Sulf2 were tested at a single concentration in HepG2 liver cancer cells to monitor effects on gene expression. The siRNAs were transfected into the cells at a concentration of 50 nM in lipofectamine RNAiMax using the manufacturer's instructions. After 24 h exposure the reduction in Sulf2 mRNA (amount of silencing) was determined by quantitative—RT-PCR (QRTPCR). The data are shown in FIG. 1 and were normalized to non-silencing control siRNA transfections (NS; set as 1.0) and compared with blank (vehicle only treated samples; Blk). The sequences selected varied in potency but, as shown in FIG. 1, SULF2_1 (SEQ ID NO:1) was among the most potent siRNAs (FIG. 1), with >90% silencing at 50 nM.

### Example 2

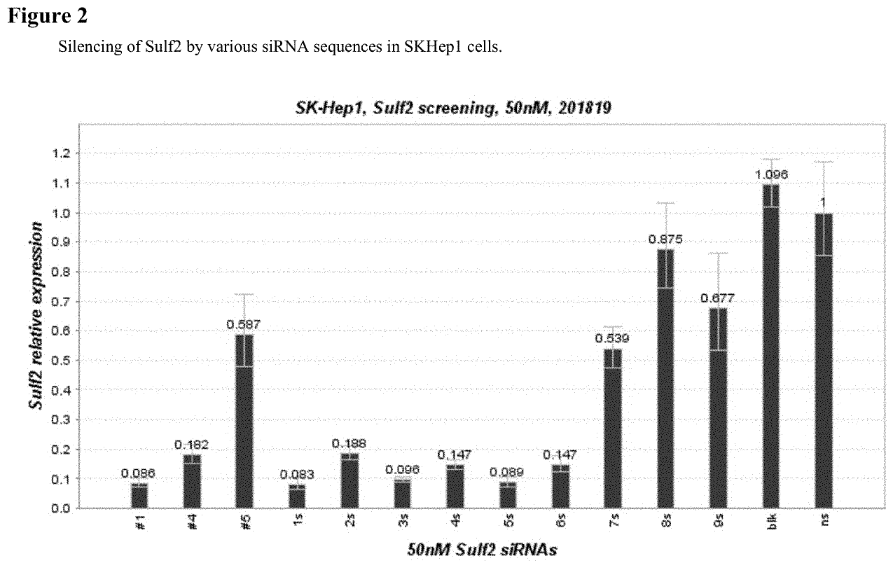

siRNA sequences against Sulf2 were tested at a single concentration in SKhep 1 liver cancer cells to monitor effects on gene expression. The siRNAs were transfected into the cells at a concentration of 50 nM in lipofectamine RNAiMax. After 24 h exposure the reduction in Sulf2 mRNA (amount of silencing) was determined by quantitative —RT-PCR (QRTPCR). The data are shown in FIG. 2 and were normalized to non-silencing control siRNA transfections (NS; set as 1.0) and compared with blank (vehicle only treated samples; Blk).

The SULF2_1 (SEQ ID NO:1) sequence again appeared to produce the greatest degree of gene silencing of SULF2 in SkHep1 cells, with >90% silencing observed at 50 nM.

### Example 3

SULF2_1 S(SEQ ID NO:1) was tested at multiple concentrations (90 nM−0.04 nM in 3 -fold dilutions) in HepG2 liver cancer cells to monitor effects on gene expression. The siRNA was transfected into the cells using lipofectamine RNAiMax. After 48 h the amount of silencing was determined by using QRTPCR. Data were normalized to non-silencing control siRNA transfections (NS; set as 1.0) and compared with blank (vehicle only treated samples; Blk).

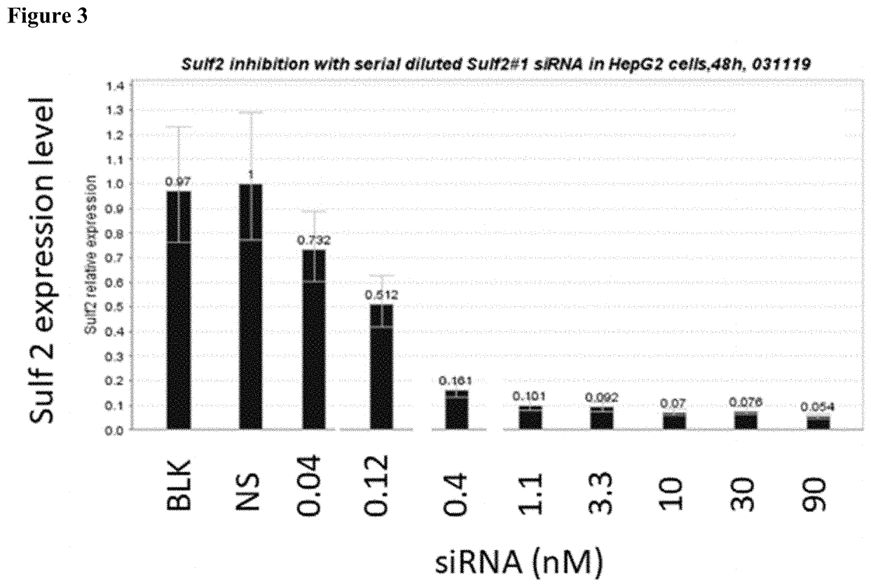

As shown in FIG. 3, potent dose dependent down regulation of SULF2 by siRNA sequence SULF2_1 was observed in HepG2 cells, and the IC₅₀ appeared to be around 0.12 nM.

### Example 4

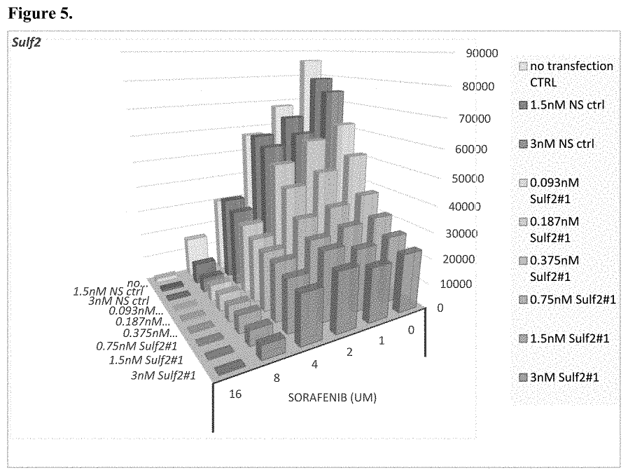

The effect of Sulf2 silencing in combination with Sorafenib in a liver cancer cell model (HepG2 cells) was studied. HepG2 cells incubated with various concentrations of SULF2_1 siRNA for 96 h, and Sorafenib (404) was added after 24 h to allow exposure for 72 h. Non-silencing siRNA (NS) was compared with SULF2_1 siRNA at various concentrations on the resulting efficacy of Sorafenib. siRNA silencing of Sulf2 alone (IC₅₀ 0.375 nM) was much more potent than Sorafenib (IC50˜4 μM) in inhibiting tumor growth in this model, as shown in FIG. 5.

### Example 5

Combinations of siRNAs were transfected into Hep3B liver cells using lipofectamine RNAiMax. Each siRNA was at a concentration of 25 nM respectively. After 72 h post-transfection, cell viability was monitored by addition of Cell TiterGlo (Promega, Madison Wis.). Samples were shaken and after incubation for 30mins at RT, the luminescence signal was measured using a Biotek Cytation plate reader fitted with luminescence optics. The luminescence signal provides a measure of the amount of ATP present in the samples which is an indicator of the number of viable cells. The data were plotted as raw luminescence values obtained from the reader. A decrease in luminescence value is associated with a decrease in viability of the cells.

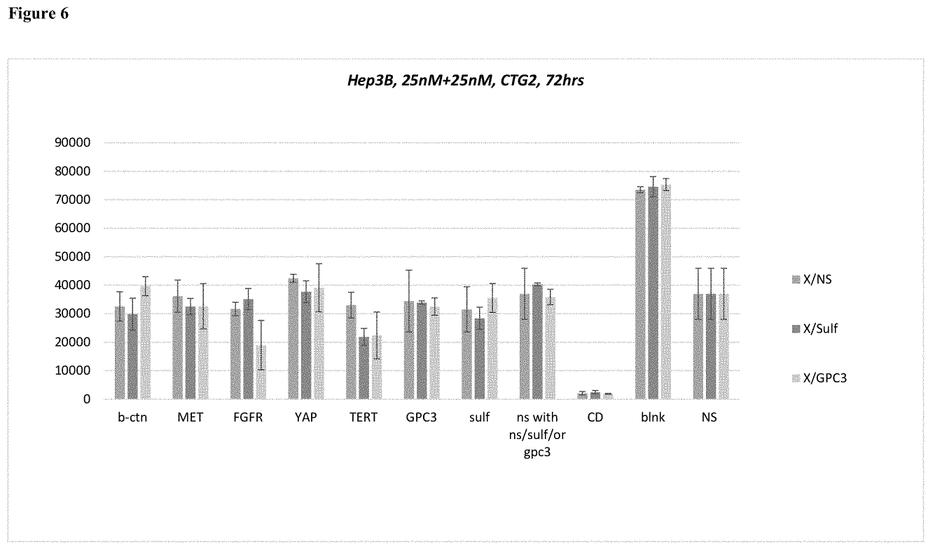

In FIG. 6, the combinations are indicated as the listed siRNA against the selected gene product. For each of β-Catenin (b-ctn), MET, FGFR, YAP, TERT, GPC3, Sulf2 and Non-silencing siRNA (ns), the orange bars represent where the siRNAs were mixed with a non-silencing siRNA (NS). The blue bars represent where the siRNAs were mixed with an siRNA against SULF2 (SULF2_1). The grey bars represent where the siRNAs were mixed with an siRNA shown to potently silence GPC3. CD=Cell Death siRNA—used to obtain maximum cell killing as a positive control.

Hep3B cells have relatively low expression levels of SULF2 compared to HepG2 or SKHEP1 cells. However, a pronounced inhibitory effect was observed when Sulf2 siRNA was combined with TERT siRNA. Furthermore, silencing GPC3 and FGFR or GPC3 and TERT also showed a greater inhibitory effect than either alone at the same final concentrations (50 nM). All values were calculated from NS ctrl.

### Example 6

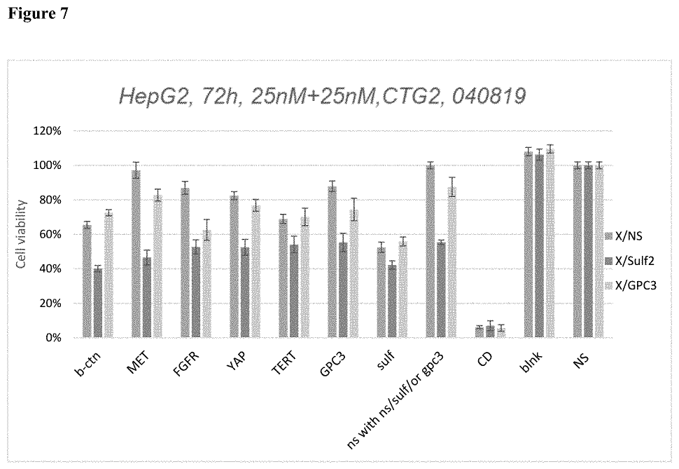

Combinations of siRNAs (each siRNA at a concentration of 25 nM) were transfected into HepG2 liver cells using lipofectamine RNAiMax. After 72 h exposure, cell viability was monitored by addition of Cell TiterGlo (Promega, Wis.). Samples were shaken and after incubation for 30 mins at RT, the luminescence signal was measured using a Biotek Cytation plate reader fitted with luminescence optics. The luminescence signal is an indication of the amount of ATP present in the samples which is an indicator of the number of viable cells. The data are shown in FIG. 7 and are plotted as the % luminescence values obtained upon exposure to non-silencing siRNA (NS). For each of β-Catenin (b-ctn), MET, FGFR, YAP, TERT, GPC3, Sulf2 and Non-silencing siRNA (ns), the orange bars represent where the siRNAs were mixed with a non-silencing siRNA (NS). The blue bars represent where the siRNAs were mixed with an siRNA against SULF2 (SULF2_1). The grey bars represent where the siRNAs were mixed with an siRNA shown to potently silence GPC3. CD=Cell Death siRNA — used to obtain maximum cell killing as a positive control. Most efficient siRNA combinations for HepG2 cells (all values are calculated from NS ctrl) were:
Sulf2+0-catenin—40%
Sulf2+Sulf2—42% (single agent works well alone)
Sulf2+MET—47%

### Example 7

Various siRNA sequences against TERT were tested at a single concentration in HepG2 liver cancer cells to monitor effects on gene expression. siRNAs were transfected into the cells at 50 nM using lipofectamine RNAiMax. After a 72 h exposure to the silencing reagents the amount of silencing was determined by using QRTPCR. Data were normalized to Blank (vehicle treated) samples.

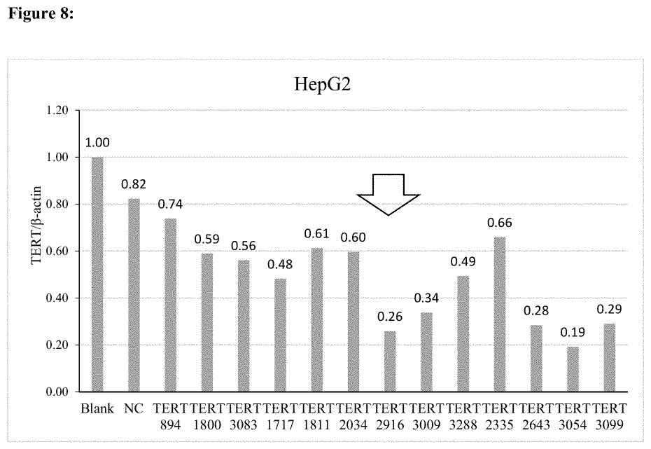

As shown in FIG. 8, TERT 2916 siRNA demonstrated silencing of TERT gene with 74% inhibition of the gene at 72 h post transfection.

### Example 8

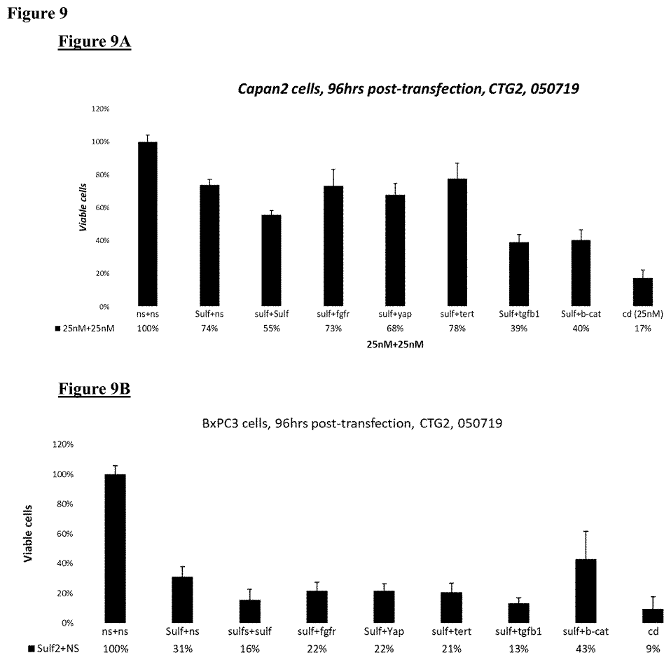

Combinations of siRNAs were tested on efficacy against cells in vitro. Combinations of siRNAs were made at 25nM+25nM and were transfected into Capan2 pancreatic cancer cells (FIG. 9a) or BxPC3 pancreatic cancer cells (FIG. 9b) using lipofectamine RNAiMax. After 96h exposure, cell viability was monitored by addition of Cell TiterGlo2 (Promega, Wis.) as described in the examples above. Data are shown for combinations with each of β-Catenin (b-ctn), MET, FGFR, YAP, TERT, GPC3, Sulf2 and Non-silencing siRNA (ns).

The data are plotted as the % luminescence values obtained upon exposure to non-silencing siRNA (NS+NS; set at 100%). The combinations with SULF2 #1 siRNA are indicated together with the % viable cells. Sulf+NS (Sulf2_1 siRNA+Non-silencing siRNA), Sulf+Sulf (Sulf2_1 siRNA at 25 nM +Sulf2_1 siRNA at 25nM), Sulf+FGFR, SULF+YAP, SULF+TERT, SULF +TGFβ1, Sulf +b-cat (β-Catenin) were mixed similarly. CD=Cell Death siRNA —used to obtain maximum cell killing as a positive control.

Treatment with Sulf2/TGFβ1 siRNAs and Sulf2/β-catenin siRNAs each reduced the number of viable Capan2 cells by 60%.

Sulf2 siRNA combinations with siRNA against TGFβ1 or β-catenin were demonstrated to have a stronger inhibitory effect than Sulf2 alone

The combination of Sulf2 siRNA together with TGFβ1 siRNA provided the greatest response in BxPC3 cells—resulting in an 87% reduction in the BxPC3 pancreatic tumor cells after 96 h.

### Example 9

### Delivery of SULF2 siRNA and TGFbetal siRNA to Tumors in vivo

SiRNAs against SULF2 and TGFβ1 were formulated into a nanoparticle using HKP(+H), a branched polypeptide containing a sequence of Histidine and Lysine residues.

BxPc3 cells were inoculated into the flank of a nude mouse to form a xenograft. Injections were given twice per week over 4 weeks. Eight animals per group were administered 1 mg/Kg intratumoral injection of Sulf2/TGFβ1 siRNA formulated in HKP(+H) at a 2.5:1 ratio (HKP(+H):siRNA). The specific treatments are shown in the table below:

| | | | | Administration |
|---|---|---|---|---|
| | | | | Route & |
| Group | Tumor | Dose | Mice | Volume |
| Control | BXPC3 | — | 8 | i.t.-50 μl; |
| | | | | twice a week |
| NC(NC siRNA/ | BXPC3 | 1 mg/kg | 8 | i.t.-50 μl; |
| HKP + H) | | | | twice a week |
| siRNAs(Sulf2 + | BXPC3 | 1 mg/kg | 8 | i.t.-50 μl; |
| TGF-β1)/HKP + H | | | | twice a week |
| siRNAs(Sulf2 + | BXPC3 | 1 mg/kg | 8 | i.t.-50 μl; |
| TERT)/HKP + H | | | | twice a week |
| siRNAs(Sulf2)/ | BXPC3 | 1 mg/kg | 8 | i.t.-50 μl; |
| HKP + H | | | | twice a week |

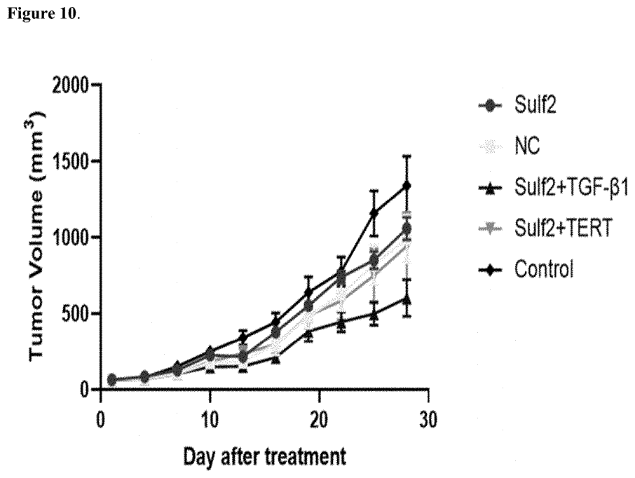

The results are shown in FIG. 10. SULF2 alone (Circles) had a small effect on tumor growth rate (compared with control (vehicle treated xenograft; diamonds). Addition of TERT siRNA with Sulf2 siRNA had little effect in this model (upside down triangles) whereas Sulf2 siRNA combined with TGFβ1 siRNA (triangles) showed a marked inhibition of tumor growth at 28 days post treatment start.

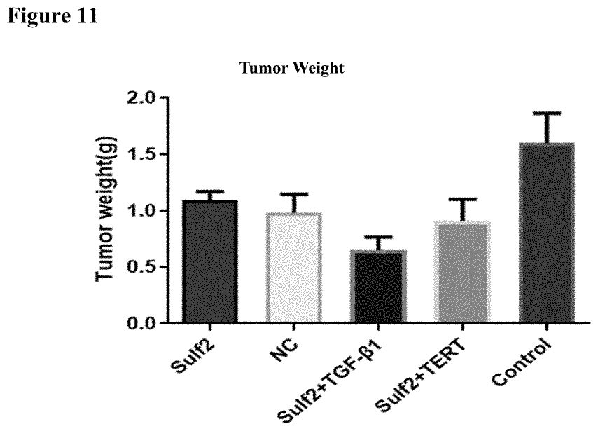

The effect of administration of siRNAs on the weight of tumor recovered from treated animals at conclusion of experiment is shown in FIG. 11A reduction in weight of the tumor when excised at the conclusion of the experiment was observed when Sulf2/TGFβ1 siRNAs were administered, compared to Sulf2 alone or NC (non-silencing control siRNA) was used.

### Example 10

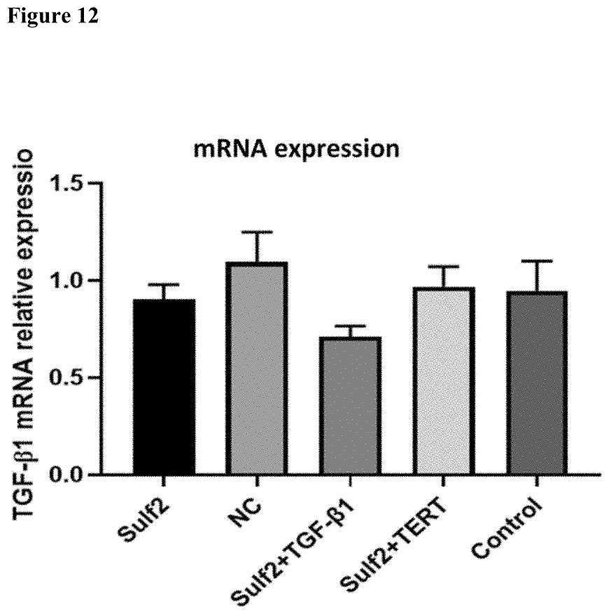

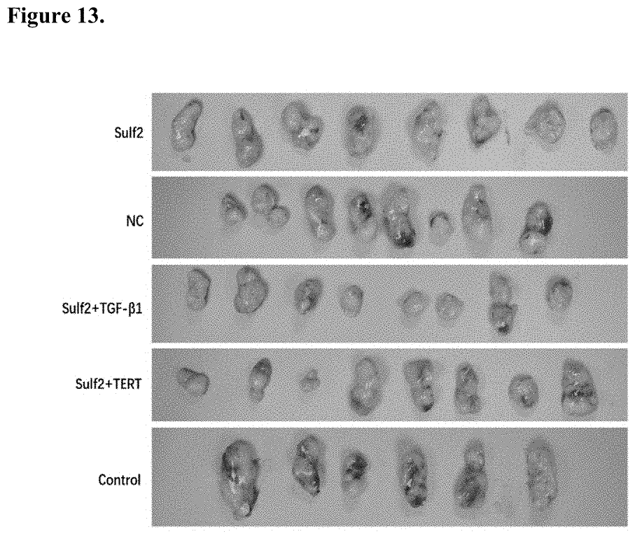

Coadministration of TGFβ/Sulf2 siRNA on TGFβ1 mRNA reduced expression in the tumor samples recovered, as shown in FIG. 12. TGFβ1 was measured using QRTPCR with primers against the gene target. Expression in the control was normalized to 1.0 and other values are represented as fractions of this signal. A reduction in TGFβ mRNA was observed upon administration of Sulf2/TGFβ1 siRNAs to the tumor. FIG. 13 shows the effect of treatment on size of resultant tumors at the conclusion of the study. After treatment of the animals with the tumor xenograft present, the animals were sacrificed and the tumors excised and photographed to show the varying sizes remaining. At the conclusion of the study SULF2+TGFβ1 siRNA treatment resulted in a marked reduction in tumor size.

1. An siRNA molecule that targets Sulf2 expression, wherein said sequence targets a Sulf2 sequence common to humans and mice.

2. The molecule according to claim 1, selected from the group consisting of molecules having the sense strand of SEQ ID NO:1-9.

3. An siRNA molecule that targets GPC3 expression, wherein said sequence targets a GPC3 sequence common to humans and mice.

4. The molecule according to claim 1, selected from the group consisting of molecules having the sense strand of SEQ ID NO:10-31.

5. A pharmaceutical composition comprising a molecule according to claim 1.

6. A pharmaceutical composition comprising a molecule according to claim 1, and further comprising a second siRNA molecule that targets a GPC3 sequence common to humans and mice.

7. A pharmaceutical composition comprising a molecule according to claim 2, and further comprising a second siRNA molecule selected from the group consisting of molecules having the sense strand of SEQ ID NO:10-31.

8. A pharmaceutical composition comprising a molecule according to claim 1, further comprising at least one siRNA that targets TGFβ1, FGFR, β-Catenin, GPC3, Yap1, MET or TERT.

9. A pharmaceutical composition comprising a molecule according to claim 2 and at least one siRNA that targets TGFβ1, FGFR, β-Catenin, GPC3, Yap1, MET or TERT.

10. The method according to claim 9, wherein said at least one siRNA that targets TGFβ1, FGFR, β-Catenin, Yap1, MET, or TERT is selected from the group consisting of molecules having the sense strand of SEQ ID NOs:32-37.

11. A pharmaceutical composition according to claim 5, wherein said composition is a nanoparticle composition.

12. The composition according to claim 11, comprising an HKP.

13. The composition according to claim 12, wherein said HKP is HKP(+H).

14. A method of treating cancer in a subject, comprising administering to the subject an effective amount of a molecule according to claim 1.

15. A method of treating cancer in a subject, comprising administering to the subject an effective amount of a composition according to claim 5.

16. The method according to claim 14, further comprising administering to said subject an effective amount of a small molecule chemotherapeutic drug.

17. The method according to claim 16, wherein said small molecule therapeutic drug is a protein kinase inhibitor.

18. The method according to claim 17, wherein said protein kinase inhibitor is sorafenib.

19. The method according to claim 14, wherein said cancer is selected from the group consisting of hepatocellular carcinoma, esophageal cancer, head and neck cancer, bladder cancer, pancreatic cancer, cholangiocarcinoma, lung cancer (NSCLC, SCLC, LUSC), colon cancer, glioblastoma, breast cancer, gastric adenocarcinomas, prostate cancer, ovarian carcinoma, cervical cancer, AML, ALL, myeloma, non-Hodgkins lymphoma, renal cancer, stomach cancer, medulloblastoma, thyroid carcinoma, rhabdomyosarcoma, osteosarcoma, squamous cell carcinoma, melanoma, leukemia, lymphoma, bladder cancer, cervical cancer, endometrial cancer, uterine cancer, and oropharyngeal cancer.

20. The method according to claim 14, wherein said composition is administered systemically or intratumorally.

21. The method according to claim 14, wherein said subject is a human.


SEQUENCE LISTING (37 sequences)

SEQ ID NO: 1 - DNA, 25 bp, Artificial Sequence
  sense strand for Sulf2 siRNA
gccgcacctttgccgtgtacctcaa

SEQ ID NO: 2 - DNA, 25 bp, Artificial Sequence
  sense strand for Sulf2 siRNA
ccaacatgctccagcggaagcgctt

SEQ ID NO: 3 - DNA, 25 bp, Artificial Sequence
  sense strand for Sulf2 siRNA
ccagaagaatgtgactgtcacaaaa

SEQ ID NO: 4 - DNA, 25 bp, Artificial Sequence
  sense strand for SULF2 siRNA
gctccagtctgcatcctttcaggaa

SEQ ID NO: 5 - DNA, 25 bp, Artificial Sequence
  sense strand for SULF2 siRNA
ggaagggcctgcaagagaaggacaa

SEQ ID NO: 6 - DNA, 25 bp, Artificial Sequence
  sense strand for SULF2 siRNA
gggcgaaagtcattggaatttttaa

SEQ ID NO: 7 - DNA, 25 bp, Artificial Sequence
  sense strand for SULF2 siRNA
cctcgcagttgtggacatttctgtt

SEQ ID NO: 8 - DNA, 25 bp, Artificial Sequence
  sense strand for SULF2 siRNA
ggacatttctgttcctgtccagata

SEQ ID NO: 9 - DNA, 25 bp, Artificial Sequence
  sense strand for SULF2 siRNA
cctttgacattttgtaaaaggccat

SEQ ID NO: 10 - DNA, 25 bp, Artificial Sequence
  GPC3 siRNA
cccgccctcgcccagcgcccaggta

SEQ ID NO: 11 - DNA, 25 bp, Artificial Sequence
  GPC3 siRNA
ccagcgcccaggtagctgcgaggaa

SEQ ID NO: 12 - DNA, 25 bp, Artificial Sequence
  GPC3 siRNA
cgcccaggtagctgcgaggaaactt

SEQ ID NO: 13 - DNA, 25 bp, Artificial Sequence
  GPC3 siRNA
gcccaggtagctgcgaggaaacttt

SEQ ID NO: 14 - DNA, 25 bp, Artificial Sequence
  GPC3 siRNA
cccaggtagctgcgaggaaactttt

SEQ ID NO: 15 - DNA, 25 bp, Artificial Sequence
  GPC3 siRNA
ccgggaccgtgcgcaccgcgtgctt

SEQ ID NO: 16 - DNA, 25 bp, Artificial Sequence
  GPC3 siRNA
gcccaacatgctgctcaagaaagat

SEQ ID NO: 17 - DNA, 25 bp, Artificial Sequence
  GPC3 siRNA
gctgctcaagaaagatggaagaaaa

SEQ ID NO: 18 - DNA, 25 bp, Artificial Sequence
  GPC3 siRNA
gctcaagaaagatggaagaaaaata

SEQ ID NO: 19 - DNA, 25 bp, Artificial Sequence
  GPC3 siRNA
gcggttttccaagaggcctttgaaa

SEQ ID NO: 20 - DNA, 25 bp, Artificial Sequence
  GPC3 siRNA
cggttttccaagaggcctttgaaat

SEQ ID NO: 21 - DNA, 25 bp, Artificial Sequence
  GPC3 siRNA
ggttttccaagaggcctttgaaatt

SEQ ID NO: 22 - DNA, 25 bp, Artificial Sequence
  GPC3 siRNA
ccaagaggcctttgaaattgttgtt

SEQ ID NO: 23 - DNA, 25 bp, Artificial Sequence
  GPC3 siRNA
ggcctttgaaattgttgttcgccat

SEQ ID NO: 24 - DNA, 25 bp, Artificial Sequence
  GPC3 siRNA
gcctgactccacaagcttttgagtt

SEQ ID NO: 25 - DNA, 25 bp, Artificial Sequence
  GPC3 siRNA
cctgactccacaagcttttgagttt

SEQ ID NO: 26 - DNA, 25 bp, Artificial Sequence
  GPC3 siRNA
ccgaggagcaagacgtgacctgaaa

SEQ ID NO: 27 - DNA, 25 bp, Artificial Sequence
  GPC3 siRNA
ggagcaagacgtgacctgaaagtat

SEQ ID NO: 28 - DNA, 25 bp, Artificial Sequence
  GPC3 siRNA
ccccaagcttattatgacccaggtt

SEQ ID NO: 29 - DNA, 25 bp, Artificial Sequence
  GPC3 siRNA
cccaagcttattatgacccaggttt

SEQ ID NO: 30 - DNA, 25 bp, Artificial Sequence
  GPC3 siRNA
gctcttactgccagggactgatgat

SEQ ID NO: 31 - DNA, 25 bp, Artificial Sequence
  GPC3 siRNA
gcaatgtggtcatgcaaggctgtat

SEQ ID NO: 32 - DNA, 23 bp, Artificial Sequence
  MET seq - may synergize with SULF2/GPC3 silencing
gcacuagcaaaguccgagadtdt

SEQ ID NO: 33 - DNA, 25 bp, Artificial Sequence
  TERT sequence - may synergize with SULF2/GPC3 silencing
ccaucagagccagucucaccuucaa

SEQ ID NO: 34 - DNA, 23 bp, Artificial Sequence
  YAP sequence - may synergize with SULF2/GPC3 silencing
ggugauacuaucaaccaaadtdt

SEQ ID NO: 35 - DNA, 23 bp, Artificial Sequence
  FGFR sequence - may synergize with SULF2/GPC3 silencing
ccaccacauugacuacuaudtdt

SEQ ID NO: 36 - DNA, 22 bp, Artificial Sequence
  B-Catenin sequence - may synergize with SULF2/GPC3 silencing
ggaccuauacuuacgaaaadtd

SEQ ID NO: 37 - DNA, 25 bp, Artificial Sequence
  TGFB sequence - may synergize with SULF2/GPC3 silencing
cccaagggcuaccaugccaacuucu